(19) United States
(12) Patent Application Publication
(10) Pub. No.: US 2022/0265753 A1
(43) Pub. Date: Aug. 25, 2022

(54) COMPOSITION FOR ALLEVIATING SYMPTOMS OF ANDROPAUSE, CONTAINING EXTRACT OF FICUS AURICULATA LOUR. AS ACTIVE INGREDIENT

(71) Applicant: KOREA RESEARCH INSTITUTE OF BIOSCIENCE AND BIOTECHNOLOGY, Daejeon (KR)

(72) Inventors: Young Bae RYU, Daejeon (KR); Sang Woo Lee, Daejeon (KR); Sang Ho Choi, Daejeon (KR); Ji Young Park, Daejeon (KR); Hyung Jun Kwon, Daejeon (KR); In Chul Lee, Daejeon (KR); Jong Sub Lee, Daejeon (KR); Woo Song Lee, Daejeon (KR); Wan Yi Li, Gonmyeong-si (CN)

(21) Appl. No.: 17/744,872

(22) Filed: May 16, 2022

Related U.S. Application Data
(62) Division of application No. 17/283,343, filed on Apr. 7, 2021, now abandoned.

(30) Foreign Application Priority Data
Oct. 8, 2018 (KR) 10-2018-0119705

(51) Int. Cl. A61K 36/60; A23L 33/105; A61P 5/24

(52) U.S. Cl. CPC A61K 36/60; A23L 33/105; A61P 5/24; A61K 2236/331; A61K 2236/333

(57) ABSTRACT

The present invention relates to a method of preventing or alleviating symptoms of andropause comprising administering a pharmaceutically effective amount of an extract of Ficus auriculata Lour. To a patient in need thereof. The extract of Ficus auriculata Lour. of the present invention exhibits the effect of preventing and alleviating erectile dysfunction by inhibiting the activity of PDE5, and the effect of preventing and alleviating prostatic hyperplasia by suppressing the expression of AR and PSA.

### FIELD OF THE INVENTION

The present invention relates to a composition for alleviating symptoms of andropause, and more particularly, to a pharmaceutical, food and food additive composition for alleviating symptoms of andropause containing extract of Ficus auriculata Lour. as an active ingredient.

### BACKGROUND OF THE INVENTION

As interest in the development of medical technology and health maintenance increases, the average life expectancy is gradually increasing, and the proportion of the population of the elderly is rapidly increasing for various social reasons. As society is aging, problems with the health of the elderly are emerging. In developed countries, it is reported that more than 40% of the elderly over the age of 85 suffer from disease, and the healthy lifespan, which indicates the period of actual active and healthy life, is used as a more important index than the average lifespan, which indicates simply being alive. Accordingly, a new market is being formed as interest in the usual health aim and pursuit of well-being leads to the purchase of health functional foods.

Benign prostatic hyperplasia(BPH) is one of the representative diseases that lower the quality of life of men along with erectile dysfunction. It occurs gradually from the 40s and 70% of men in their 70s suffer, and the number is increasing every year. Erectile dysfunction is also known to have a high prevalence. According to the National Health Information Portal, erectile dysfunction occurs in 64% of people over 50s and 86% of over 60s. According to the Korean Urological Association, the number of erectile dysfunction patients is estimated to be about 2.3 million. The reciprocal prevalence between BPH and erectile dysfunction has already been mentioned in many studies, and the Korean Urology Association found that more than 80% of erectile dysfunction patients in Korea are accompanied by BPH. Penile erection is a complex physiological reaction that occurs due to the comprehensive actions of blood vessels, endocrine system, and nervous system. As the cavernous smooth muscle is relaxed by various stimuli, the pore swells, and the intra-penis pressure increases due to the increase in blood flow due to arteriole dilatation, the subtunical veins which present between the relatively hard albuginea and the pores are pressed by the expansion of the pores, and the leakage of venous blood is blocked. As a result, the intra-penis pressure further increases, leading to an erection. As the physiological phenomena of penile erection have been revealed and the pharmacological action and mechanism of various drugs on the cavernous smooth muscle have been studied, efforts to use drugs that have a relaxing effect on the cavernous smooth muscle for the treatment of erectile dysfunction are emerging. Currently, substances that relax the cavernous smooth muscle include adrenergic α-receptor blockers, cholinergic drugs, NO (Nitric oxide), peptides, prostaglandins, histamines, calcium channel blockers, potassium channel openers, and nonspecific vasodilators. BPH (benign prostatic hyperplasia), a typical male disease, is a disease in which the prostate is abnormally enlarged and occurs in 50% of men over 50, 60% of men aged 60, and 90% of men aged 85, and refers to a symptom of urinary disorders accompanied by symptoms such as frequent urinationn, residual urine, nocturia, urethra, urinary urgency, weak urine, and urinary hesitation, as the urethral resistance increases due to a hypertrophied prostate. The exact pathogenesis of benign prostatic hyperplasia has not been identified yet, but the hypothesis that testosterone in the blood is converted to dihydrotestosterone (DHT) by 5-α-reductase in the prostate and androgen receptor (AR) is stimulated by DHT to induce benign prostatic hyperplasia is dominant. Currently, drugs suitable for the treatment of both diseases of benign prostatic hyperplasia and erectile dysfunction are being prescribed. In the case of BPH, according to the treatment guidelines, alpha blockers, 5ARI, anticholinergic drugs, and tadalafil, a PDE-5 inhibitor, are prescribed in combination. Basically, a lot of alpha-blockers are prescribed for the treatment of urination disorders caused by BPH, and among them, tamsulosin-based formulations with less side effects such as orthostatic hypotension are most often used. In the case of erectile dysfunction, PDE-5 inhibitor drugs such as sildenafil and tadalafil are mainly prescribed. Therefore, patients with both diseases are often prescribed a combination of erectile dysfunction drugs, alpha blockers, and 5ARI. Tadalafil 5mg is an erectile dysfunction drug and has indications for BPH. In particular, tadalafil 5mg is also approved as a daily therapy (once a day), so it has the advantage of being able to treat normal sex life and urination disorders due to enlarged prostate at once when taken daily. However, tadalafil 5mg is less effective in treating severe BPH. Therefore, when suffering from erectile dysfunction and severe BPH, tadalafil 5mg and alpha blockers should be taken together. However, if you take both drugs together, the rate of taking the drugs well and in time decreases, and there is a burden of increasing the cost. Accordingly, the inventors of the present invention solved the concern of addiction or side effects by using natural substances as raw materials, and found an ingredient having an effect of simultaneously alleviating both diseases of erectile dysfunction and BPH, and attempted to develop a composition capable of preventing and treating various diseases caused by andropause.

As related documents, patent documents WO2012032494 A1 and “Clinical study on erectile dysfunction in diabetic and non-diabetic subjects and its management with Ficus relegiosa Linn.” Ayu. 2010 Jul.-Sep.; 31(3):272-279.

### DETAILED DESCRIPTION OF THE INVENTION

### Summary

An object of the present invention is to provide a composition for alleviating symptoms of andropause that inhibits PDE5, androgen receptor (AR) and prostate specific antigen (PSA) expression.

Also, an object of the present invention is to provide a composition that contains extract of Ficus auriculata Lour. as an active ingredient, and is effective in erectile dysfunction, BPH, and alopecia.

Furthermore, an object of the present invention is to provide a new preventive and therapeutic pharmaceutical composition, food composition, and food additive for alleviating symptoms of andropause containing extract of Ficus auriculata Lour. as an active ingredient.

### Technical Solution

The present invention provides a pharmaceutical composition for preventing or alleviating symptoms of andropause containing extract of Ficus auriculata Lour. as an active ingredient.

The extract of Ficus auriculata Lour. includes leaves, fruits, stems, and roots, and preferably, a fruit extract is used. Also, extraction may be performed by hot water extraction, solvent extraction, or ultrasonic extraction method, preferably hot water extraction or solvent extraction, and in the case of solvent extraction, ethanol is preferably used.

In the present invention, the symptoms of andropause is a combination of symptoms such as lower urinary tract symptoms, BPH, prostatitis, nervous irritability, emotional anxiety, depression, hot flashes, sleep disorders, hypodynamia, reduced work ability, erectile dysfunction, and alopecia, and refers to a case of one or more of the above symptoms. Improvement of these symptoms is achieved by inhibition of PDE5 activation, androgen receptor expression, or PSA (Prostate specific antigen) expression, and the composition provided by the present invention improves symptoms of andropause by the above mechanism.

Also, the present invention provides a food composition and food additives for alleviating symptoms of andropause containing extract of Ficus auriculata Lour. as an active ingredient.

### Effects of the Invention

The extract of Ficus auriculata Lour. of the present invention has no cytotoxicity, inhibits the activity of PDE5, and inhibits the production of androgen receptors and prostate-specific antigens to exhibit an improvement and prevention effect on erectile dysfunction, BPH, and alopecia. Therefore the composition containing the extract of Ficus auriculata Lour. of the present invention can be usefully used as a medicine, food, food additive, and the like.

### BRIEF DESCRIPTION OF THE DRAWINGS

FIG. 1 is a diagram confirming the cytotoxicity of an extract of Ficus auriculata Lour. against prostate cancer cell line (LNCaP).

FIG. 2 is a diagram illustrating the effect of the fruit extract of Ficus auriculata Lour. by country of origin on inhibiting the production of testosterone-induced prostate-specific antigen (PSA) against prostate cancer cell lines (LNCaP).

FIG. 3 is a diagram illustrating the effect of the extract for each portion of Ficus auriculata Lour. on inhibiting the production of testosterone-induced prostate-specific antigen (PSA) against prostate cancer cell lines (LNCaP).

FIG. 4 is a diagram illustrating the effect of the extract of Ficus auriculata Lour. by the concentration on inhibiting the production of testosterone-induced prostate-specific antigen (PSA) against prostate cancer cell lines (LNCaP).

FIG. 5 is a diagram illustrating the effect of the fruit extract of Ficus auriculata Lour. on inhibiting the production of androgen receptor (AR) in prostate cancer cell lines (LNCaP).

FIG. 6 is a diagram illustrating the enzyme inhibitory activity of an extract of Ficus auriculata Lour. against PDE5, an enzyme involved in erectile dysfunction.

### DETAILED DESCRIPTION

The present invention provides a pharmaceutical composition for preventing or alleviating symptoms of andropause containing extract of Ficus auriculata Lour. as an active ingredient.

However, the following Examples, Experimental Examples, and Preparation Examples are merely illustrative of the present invention, and the contents of the present invention are not limited to the following Examples, Experimental Examples, and Preparation Examples.

### Example 1 Preparation of Extracts of Ficus auriculata Lour.

The extract of Ficus auriculata Lour. used in the present invention was prepared and prepared as follows.

Ethanol was added to each dried product of leaves, fruits, and stems obtained by applying for pre-sale from the International Biological Materials Research Center of the Korea Research Institute of Bioscience and Biotechnology, and the extracts were prepared by immersion and concentration under reduced pressure.

### Example 2 Cell Culture

Prostate cancer cell line (LNCaP cell) was cultured in RMPI-1640 medium containing 10% fetal bovine serum (FBS). Prostate cancer cell lines were placed in a 175 cm² plastic flask (SPL life science Co., Ltd. Korea) and were cultured in RPMI-1640 medium containing 10% FBS and 1% antibiotic/antimycotics at 37° C. and 5% CO₂.

Cell lines were maintained by secondary culture once every 2-3 days.

### Example 3 Cell Quantification

After removing the medium solution from the 175 cm² plastic flask in which the cells were grown, washing with CMF-PBS (calcium magnesium free-phosphate buffered saline, pH 7.2), the cells were removed from the bottom of the flask by treatment with 0.25% trypsin/EDTA, neutralized with a cell culture solution, and centrifuged (1000 rpm, 3 min). A culture solution was added to the remaining cell pellet, and then a single cell suspension was prepared by repeatedly aspirating with a sterile pipette. The prepared cell suspension and trypan blue were mixed at a ratio of 1:1 and measured using a hemocytometer on an optical microscope.

### Experimental Example 1 Confirmation of Cell Viability of Ficus auriculata Lour

Toxicity to the prostate cancer cell line (LNCaP cell) of Ficus aurikulata Lour. was measured using the EzCytox kit. Specifically, the prostate cancer cell line was dispensed into a 96 well plate at 1×10⁴ cells/well. This was cultured in an incubator under conditions of 37° C. and 5% CO₂, and then incubated for 24 hours by adding Ficus aurikulata Lour. of various concentrations of 0, 6.25, 12.5, 25, 50, 100, 200, 400 μg/ml. The prostate cancer cell line (LNCaP cell) cultured for 24 hours were reacted for 1 hour using the EzCytox kit, and then the absorbance was measured. The cytotoxicity of Ficus auriculata Lour. was determined by calculating the cell viability based on the control.

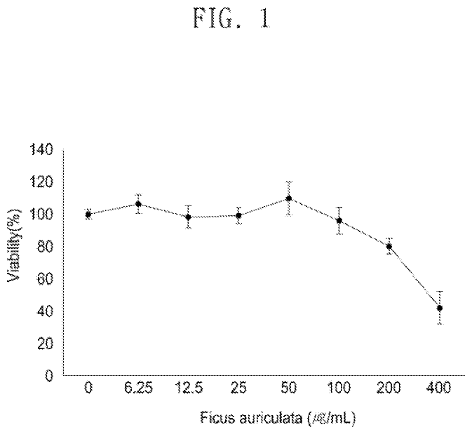

As a result, as shown in FIG. 1, when Ficus auriculata Lour. was not added, the prostate cancer cell line (LNCaP cell) exhibited a survival rate of 100%, and in the group to which Ficus auriculata Lour. was added at various concentrations, the survival rate was more than 90% up to the concentration of 100 μg/ml. Therefore, it was confirmed that Ficus auriculata Lour. of the present invention did not exhibit cytotoxicity against prostate cancer cell lines (LNCaP cell) (see FIG. 1).

### Experimental Example 2 Confirmation of the Inhibitory Effect of the Fruit Extract of Ficus auriculata Lour. by Country of Origin Against Testosterone-Induced Prostate Specific Antigen (PSA)

Reverse transcription-real-time polymerase chain reaction (real-time RT-PCR) was used to measure the inhibition of expression of prostate-specific antigen (PSA) in testosterone-induced prostate cancer cell lines (LNCaP cells). Specifically, the prostate cancer cell line (LNCaP cells) was dispensed to a 6 well plate at 5×10⁵ cells/well. After incubating for 24 hours in an incubator under conditions of 37° C. and 5% CO₂, Ficus auriculata Lour. fruit extract of each country of origin (Vietnam, Nepal, Myanmar, Bangladesh, China, Laos) was added at 100 μg/ml and incubated for 3 hours. Testosterone 100 nM was added to each well except for the control and incubated for 24 hours. After incubation for 24 hours, the supernatant was removed, total RNA was extracted using Trizol, and cDNA was synthesized using PrimeScript RT Master Mix (Takara, Japan). The synthesized cDNA was subjected to real-time polymerase chain reaction using TOPreal qPCR 2X PreMIX (Enzynomics, Korea). The primers and PCR conditions used are shown in Tables 1 and 2.

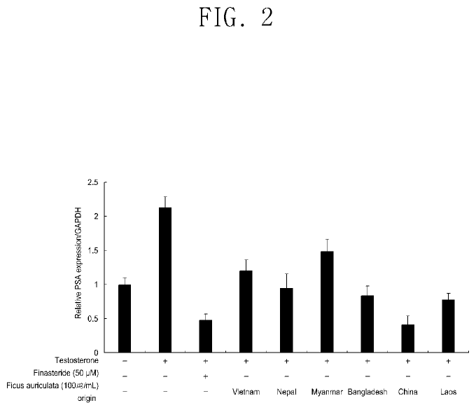

As a result, as shown in FIG. 2, it was confirmed that in the group treated with 100 μg/ml of the fruit extract of Ficus auriculata Lour. produced in China, the PSA inhibitory ability was 80% higher than that of the group treated with only testosterone. (See FIG. 2)

### Experimental Example 3 Confirmation of the Inhibitory Effect of Ficus auriculata Lour. on Testosterone-Induced Prostate Specific Antigen (PSA) by Site

Reverse transcription-real-time polymerase chain reaction (real-time RT-PCR) was used to measure the inhibition of expression of prostate-specific antigen (PSA) in testosterone-induced prostate cancer cell lines (LNCaP cells). Specifically, the prostate cancer cell line (LNCaP cells) was dispensed to a 6 well plate at 5×10⁵ cells/well. After incubating for 24 hours in an incubator under conditions of 37° C. and 5% CO₂, extracts of each part of Ficus auriculata Lour. (leaf, fruit, stem) were added at 100 μg/ml and incubated for 3 hours. Testosterone 100 nM was added to each well except for the control and incubated for 24 hours. After incubation for 24 hours, the supernatant was removed, total RNA was extracted using Trizol, and cDNA was synthesized using PrimeScript RT Master Mix (Takara, Japan). The synthesized cDNA was subjected to real-time polymerase chain reaction using TOPreal qPCR 2X PreMIX (Enzynomics, Korea). The primers and PCR conditions used are shown in Tables 1 and 2.

TABLE 1

| Gene | Hot start | Denaturation | Annealing |
|---|---|---|---|
| Prostate specific | 95° C. | 95° C. | 60° C. |
| antigen (PSA) | 12 min | 10 sec | 30 sec |
| Androgen receptor | | | |
| (AR) | | | |
| GAPDH | | | |

TABLE 2

| Gene | Primer | |
|---|---|---|
| Prostate specific | Forward | CAAGTTCATGCTGTGTGCTGGA |
| antigen (PSA) | Reverse | AGGGCTGGCATGGTCAGTTC |
| Androgen receptor | Forward | TCCATTGCCCACCAAAGACTA |
| (AR) | Reverse | GCAAATCTGGCCTGTCACCTC |
| GAPDH | Forward | GCACCGTCAAGGCTGAGAAC |
| | Reverse | TGGTGAAGACGCCAGTGGA |

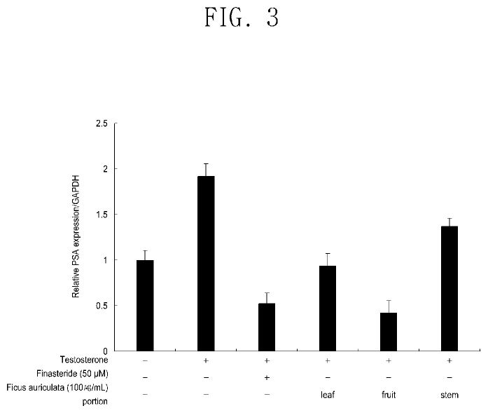

As a result, as shown in FIG. 3, in prostate cancer cell lines, PSA expression was increased by 190% by testosterone, and PSA inhibitory effect was found to be 80%, 50%, and 30% respectively in the order of fruit, leaf, and stem extracts compared to the testosterone-treated group.

### Experimental Example 4 Confirmation of the Inhibitory Effect by Concentration of Ficus auriculata Lour. Fruit Extract Against Testosterone-Induced Prostate Specific Antigen (PSA).

Reverse transcription-real-time polymerase chain reaction (real-time RT-PCR) was used to measure the inhibition of expression of prostate-specific antigen (PSA) in testosterone-induced prostate cancer cell lines (LNCaP cells). Specifically, the prostate cancer cell (LNCaP cells) line was dispensed to a 6 well plate at 5×10⁵ cells/well. After incubating for 24 hours in an incubator under conditions of 37° C. and 5% CO₂, 0 (control), 12.5, 25, 50, 100 μg/ml of various concentrations of Ficus auriculata Lour. fruit extracts were added and incubated for 3 hours. Testosterone 100 nM was added to each well except for the control and incubated for 24 hours. After incubation for 24 hours, the supernatant was removed, total RNA was extracted using Trizol, and cDNA was synthesized using PrimeScript RT Master Mix (Takara, Japan). The synthesized cDNA was subjected to real-time polymerase chain reaction using TOPreal qPCR 2X PreMIX (Enzynomics, Korea). The primers and PCR conditions used are shown in Tables 1 and 2.

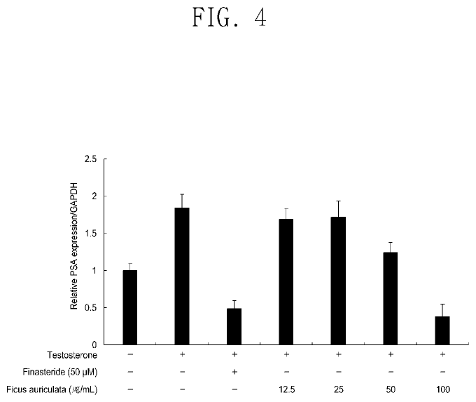

As a result, it was confirmed that, as shown in FIG. 4, when not treated with Ficus auriculata Lour., the expression of PSA was increased by 180% by testosterone in prostate cancer cell lines (LNCaP cells). Compared to the testosterone-treated group, when 50 μg/ml and 100 μg/ml of Ficus auriculata Lour. were added, 30% and 70% of PSA expression inhibitory ability was shown, respectively. (See FIG. 4)

### Experimental Example 5 Confirmation of the Effect of Inhibiting the Expression of Androgen Receptor (AR) of Ficus auriculata Lour. Fruit Extract

Reverse transcription-real-time polymerase chain reaction (real-time RT-PCR) was used to measure androgen receptor (AR) expression in prostate cancer cell lines (LNCaP cells).

Specifically, the prostate cancer cell line (LNCaP cells) was dispensed to a 6 well plate at 5×10⁵ cells/well. After incubating for 24 hours in an incubator under conditions of 37° C. and 5% CO₂, 0 (control), 12.5, 25, 50, 100 μg/ml of various concentrations of Ficus auriculata Lour. fruit extracts were added and incubated for 24 hours. After incubation for 24 hours, the supernatant was removed, total RNA was extracted using Trizol, and cDNA was synthesized using PrimeScript RT Master Mix (Takara, Japan). The synthesized cDNA was subjected to real-time polymerase chain reaction using TOPreal qPCR 2X PreMIX (Enzynomics, Korea). The primers and PCR conditions used are shown in Tables 1 and 2.

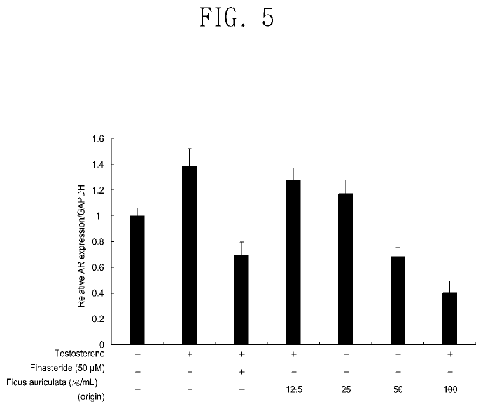

As a result, it was confirmed that, as shown in FIG. 5, when the fruit extract of Ficus auriculata Lour. was not treated, the expression of AR was increased by 140% by testosterone in prostate cancer cell lines (LNCaP cells), and when 50 μg/ml and 100 μg/ml of Ficus auriculata Lour. fruit extract was added, it was confirmed that the AR expression inhibitory effect of 50% and 70% was shown, respectively. (See FIG. 5)

Additionally, Androgen Receptor (AR) is known to play an important role in Androgenetic Alopecia (male pattern baldness) (J Endocrinol. 1998 Jan.; 156(1):59-65, Arch Dermatol Res. 2012 Sep.; 304(7):499-510.), and in particular, higher AR is expressed than normal hair papilla cells in the alopecia area hair papilla cells of alopecia patients, and it has been reported that suppressing this can prevent the progression of alopecia. (J Endocrinol. 1998 Jan.; 156(1):59-65, The Matabolic Basis of Inherited Disease. McGrwa-Hill; New York:1989. pp. 1919-1944). Also, AR is known to play an important role in hirsutism and beard growth caused by increased body hair in normal areas. (Arch Dermatol Res. 2012 Sep.; 304(7):499-510, Journal of cutaneous medicine and surgery, 3(1), 9-15). Therefore, inhibiting AR in body hair and hirsutism can relatively inhibit hair growth. (Journal of cutaneous medicine and surgery, 3(1), 9-15, Fertility and sterility 64.3(1995):511-517).

The fruit extract of Ficus auriculata Lour. of the present invention can exhibit the effect of inhibiting alopecia, which is one of the representative menopausal symptoms, by inhibiting AR.

### Experimental Example 6 Confirmation of the Inhibitory Effect of PDE5A1 Enzyme of Ficus auriculata Lour.

In order to measure the inhibitory activity of the sample against PDE5A1, 10 μl of the assay buffer (10 mM Tris-HCl, pH 7.4, 10 mM MgCl₂, 0.01 mg/ml BSA, 0.05% Tween20) was added to a 96-well black plate, and 5 μl of the sample diluted by 2 times(Final concentration 0˜200 μg/ml) using the assay buffer was added to each well. After adding 20 μl of 200 μg/ml of PDE5A1 enzyme to each well and mixing 15 μl each of 20 μM FAM-Cyclic-3′,5′-GMP as a substrate, the fluorescence values are measured at intervals of 2 minutes for 30 to 60 minutes in kinetic mode at excetation 475-495 nm and emission 518-538 nm using a microplater reader.

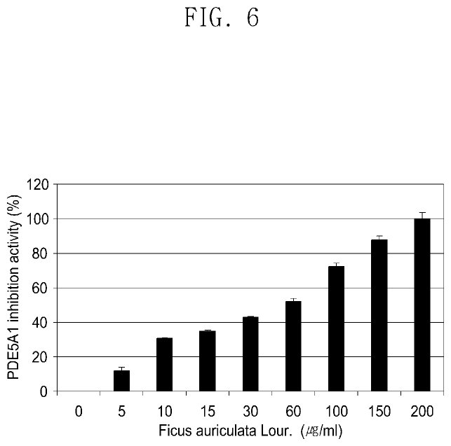

As a result, as shown in FIG. 6, inhibitory activity of PDE5A1 by Ficus auriculata Lour. was 43.2%, 52.5% and 72.6% respectively when 30 μg/ml, 60 μg/ml and 100 μg/ml were added, and showed an IC₅₀ of 49.6 μg/ml. Sildenafil citrate used as a positive control was 3.5 nM, and Rolipram had an IC₅₀ value of 114.5 μM.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

The composition provided by the present invention has no cytotoxicity, inhibits the activity of PDE5, and inhibits the production of androgen receptors and prostate-specific antigens to improve and prevent erectile dysfunction, prostatic hyperplasia, and alopecia. Therefore it has high industrial applicability as food additives, etc.

What is claimed is:

1. A method of preventing or alleviating symptoms of andropause comprising administering a pharmaceutically effective amount of an extract of Ficus auriculata Lour. to a patient in need thereof.

2. The method of claim 1, wherein the extract is a fruit extract of Ficus auriculata Lour.

3. The method of claim 1, wherein the symptoms of andropause is one or more selected from the group consisting of lower urinary tract symptoms, BPH, prostatitis, nervousness, emotional anxiety, depression, hot flashes, sleep disorders, decreased vitality, reduced work ability, erectile dysfunction, and alopecia.

4. The method of claim 1, wherein the extract is a hot water extract or ethanol extract

5. The method of claim 1, wherein the symptoms of andropause is caused by activation of PDE5, androgen receptor expression, or PSA (Prostate specific antigen) expression.
